(19) United States
(12) Patent Application Publication
(10) Pub. No.: US 2013/0218139 A1
(43) Pub. Date: Aug. 22, 2013

(54) CRIMPING DEVICE AND METHOD FOR MOUNTING A VASCULAR STENT

(71) Applicant: BIOTRONIK AG, (US)

(72) Inventor: Amir Fargahi, Buelach (CH)

(73) Assignee: BIOTRONIK AG, Buelach (CH)

(21) Appl. No.: 13/752,892

(22) Filed: Jan. 29, 2013

Related U.S. Application Data
(60) Provisional application No. 61/599,431, filed on Feb. 16, 2012.

(51) Int. Cl. A61B 17/00; A61F 2/844

(52) U.S. Cl. CPC A61B 17/00; A61F 2/844
USPC 606/1

(57) ABSTRACT

A crimping device for mounting a vascular stent, or a heart valve prosthesis introduced in a vascular stent, on an insertion catheter, comprising a forming tool having a rotationally symmetrical forming cavity, which at one end comprises a stent receiving section, at the opposing end a stent outfeed section and in between these a tapering section, and a pushing tool, which comprises a retaining and handling section and a stent forming section, wherein the latter is designed to penetrate the forming cavity of the forming tool from the open end of the stent receiving section and to seize and push back the vascular stent introduced there through the tapering section.

### CROSS REFERENCE

The present application claims priority on co-pending U.S. Provisional Application No. 61/599,431 filed on Feb. 16, 2012; which application is incorporated herein by reference.

### TECHNICAL FIELD

One aspect of the invention relates to a crimping device for mounting a vascular stent, or a heart valve prosthesis that is introduced in a vascular stent, on an insertion catheter. The invention further relates to a method for mounting a vascular stent, or a heart valve prosthesis that is introduced in a vascular stent, on an insertion catheter.

### BACKGROUND

Vascular stents (vessel supports) that are inserted in damaged vessels by means of an insertion catheter so as to lastingly safeguard blood flow have been known for quite some time and are used widely in clinical applications. Vascular stents are known to be mounted on the insertion catheter in a state in which they have a reduced diameter and to be introduced together with this catheter and in the implanted state, which is achieved, for example, by dilating an inflated balloon located inside them or by memory effects, they assume the expanded usage state thereof in the patient's body.

Of late, heart valve prosthesis have become known, which can be implanted using minimally invasive procedures and in which the actual heart valve is provided in a vascular stent that has the aforementioned typical usage characteristics. With such heart valve prosthesis, such as the products “Corevalve” from Medtronic Inc. or “Sapien” from Edwards Life Sciences Inc., the mounting on the insertion catheter requires special tools and handling. The known tools have a relatively complicated design and are complicated to handle, whereby the mounting of the prosthesis on the insertion catheter becomes a relatively cumbersome and also costly process.

### SUMMARY

It is therefore one object of the invention to provide an improved crimping device and an improved method for mounting a vascular stent, or a heart valve prosthesis that is introduced in a vascular stent, on an insertion catheter, which are notably uncomplicated and cost-effective.

This object is achieved in terms of the device aspect by a crimping device having the characteristics of claim 1 and in terms of the method aspect by a method having the characteristics of claim 11. Advantageous refinements of the inventive idea are the subject matter of the dependent claims.

One embodiment of the invention includes the consideration of carrying out the mounting of the prosthesis on the insertion catheter, which typically comprises an inner tube and an outer tube that can be displaced with respect to the same, in one continuous operation which comprises reducing the prosthesis diameter and sliding the prosthesis between the inner and outer tubes of the insertion catheter. It further includes the design of providing a device for this purpose, which automatically reduces the prosthesis diameter due to geometric configuration thereof in a single simple operating step. Moreover, it includes the consideration of providing a special tool for carrying out this operating step, the tool being adapted to the diameter-reducing geometric configuration of the device. Moreover, the desired simple operation requires that both the prosthesis and the section of the insertion catheter in which it is to be introduced are fixed in the device in advance or (in terms of the device aspects) that the device is designed for such fixation.

According to the foregoing description, an embodiment of the device comprises a forming tool having a rotationally symmetrical forming cavity, which at one end comprises a stent receiving section, at the opposing end an outer tube receiving section and between these ends a tapering section. Moreover, it comprises a pushing tool, which comprises a retaining and handling section and a stent forming section, wherein the latter is designed to penetrate the forming cavity of the forming tool from the open end of the stent receiving section and to seize and push back the vascular stent introduced there through the tapering section.

In one embodiment of the invention, the stent forming section of the pushing tool comprises a plurality of flexible rods, or ribs that are tapered toward the free end thereof, and the retaining and handling section comprises a circular ring-shaped or disk-shaped holder, on the circumference of which the rods are held on one side, in particular at identical angular distances. The number of rods or ribs should be sufficiently high so as to ensure that, over the circumference, the prosthesis can be pushed evenly through the tapering section of the forming tool. For this purpose, three rods or ribs may be sufficient; from the present view, however, a design having four or more rods or ribs is preferred.

According to a further embodiment, the forming tool has a multi-piece design, notably a two-piece design and can be folded open. The forming tool can, in principle, also have a single-piece design, or it may be composed of three or more parts, wherein the specific design is selected depending on the special geometric configuration of the stent, or of the heart valve prosthesis, and cost considerations. From the present view, a two-piece design appears to be a good compromise between high usage value and acceptable costs; and a design that can be folded open is particularly easy and reliable to operate.

According to a further embodiment, the outer tube receiving section of the forming tool has a larger diameter than the adjoining end of the tapering section such that the end of the outer tube receiving section forms an annular stop for the distal end of the outer tube of the insertion catheter.

In a further embodiment, the tapering section of the forming tool has a truncated cone shape. In a modification of this design, which is advantageous in particular for specially shaped stents, the tapering section of the forming tool is composed of two truncated cone-shaped sections having differing cone angles.

Moreover, the wall of the tapering section of the forming tool can be curved. The latter design allows special deformation profiles or usage configurations to be produced in a very deliberate manner.

In a further embodiment, the forming tool is produced from transparent material at least in some sections. This embodiment advantageously allows visual control of the mounting or crimping process without interim opening of the tool and the disadvantages associated therewith. Possible materials include Plexiglass or another highly transparent plastic; moreover, the forming tool can be produced from plastic materials such as polycarbonate or Grilamid TR 55. These materials are generally suited for producing the pushing tool, however metals (for example stainless steel) can also be used the rods or ribs thereof.

In a further embodiment, the contact surfaces are provided with a hydrophobic or hydrophilic coating so as to reduce the friction between the forming tool and the pushing tool.

Advantageous embodiments of the method will essentially be apparent directly from the aforementioned device aspects, so that these need not be repeated here.

Advantages and functional characteristics of the invention will additionally become apparent hereafter from the description of exemplary embodiments based on the figures. In the drawings:

### DESCRIPTION OF THE DRAWINGS

FIGS. 1A and 1B show perspective illustrations of the forming tool of one embodiment of the device according to the invention, in the semi-open and closed states;

FIG. 2 is a schematic illustration of one embodiment of the pushing tool; and

FIGS. 3A to 3D are schematic illustrations of one embodiment of the device according to the invention in various phases of the mounting process.

### DETAILED DESCRIPTION

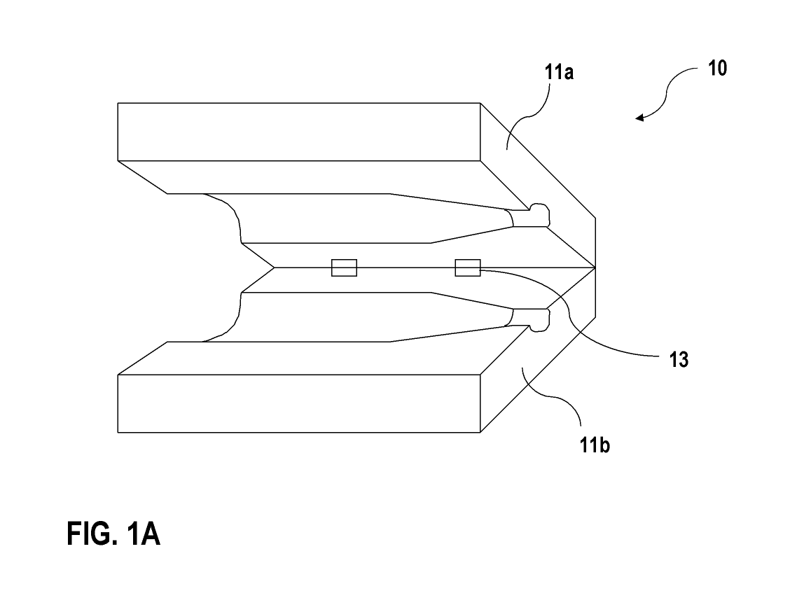

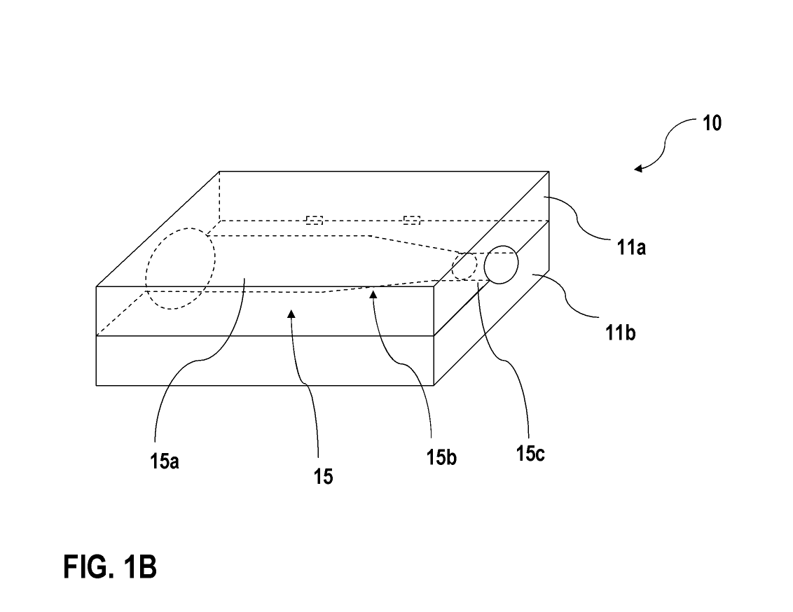

FIGS. 1A and 1B show a forming tool 10 of a crimping device according to the invention, the forming tool being composed of two identical half shells 11a, 11b having folding hinges 13, in the semi-open state (FIG. 1A) and in the closed state (FIG. 1B). In the closed usage state, identical shapes in the two half shells 11a, 11b create a forming cavity 15, which comprises a stent receiving section 15a, a tapering section 15b and an outer tube receiving section 15c. The functions of the individual sections of the forming cavity will become clear hereafter from the description of a modified design.

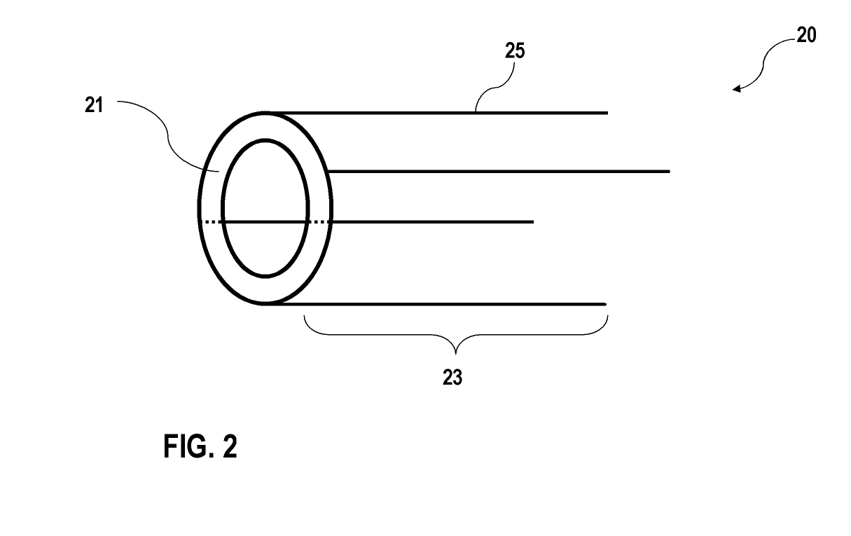

FIG. 2 shows schematically the design of a pushing tool 20, which is part of a crimping device according to the invention. It comprises a retaining and handling section 21 designed as a circular ring and a stent forming section 23, which is formed by four flexible rods 25 that are attached on one side to the retaining and handling section 21 at identical angular distances. Other numbers of rods 25 are present in other embodiments, with examples include 3, 5 and others.

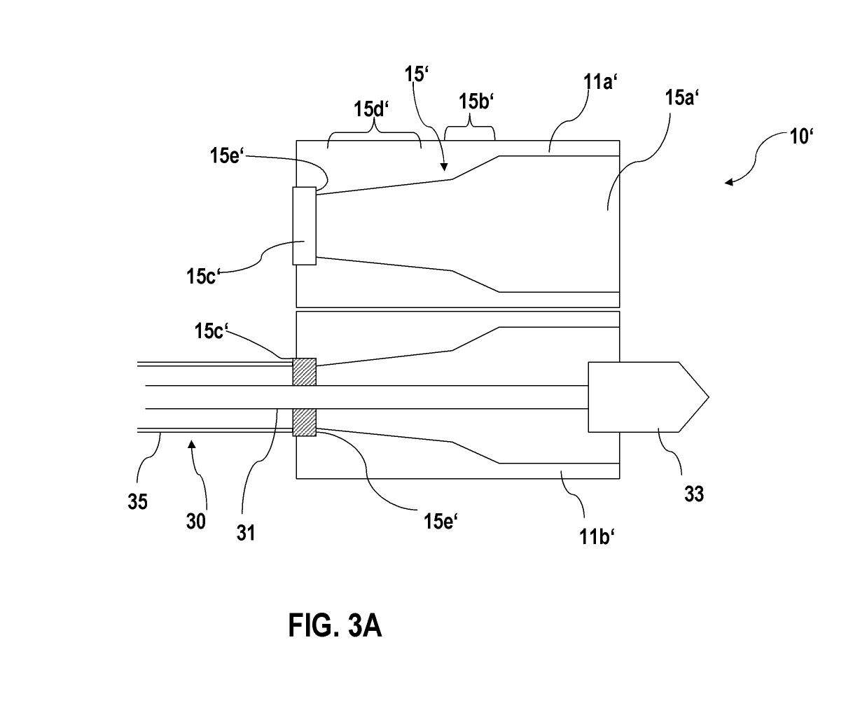

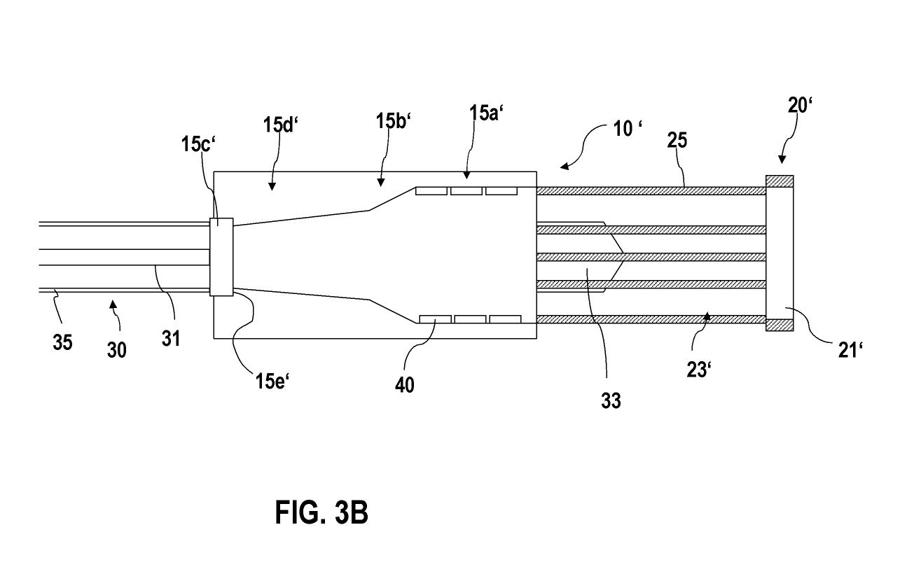

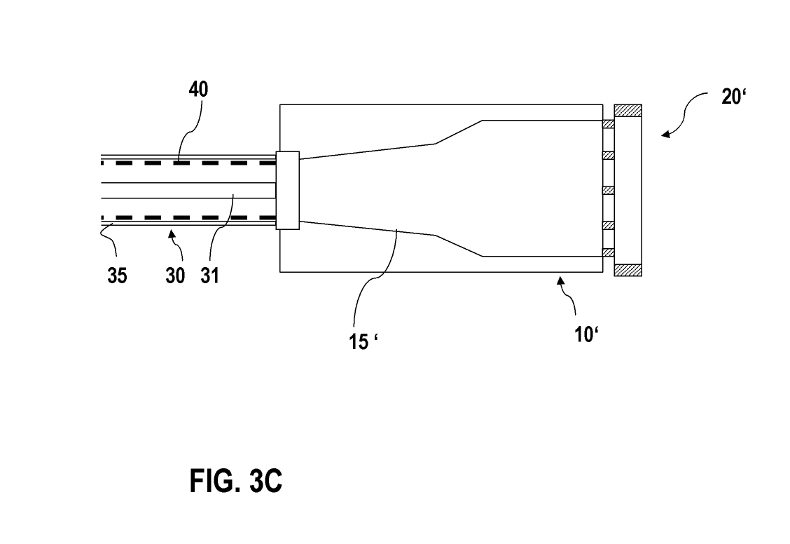

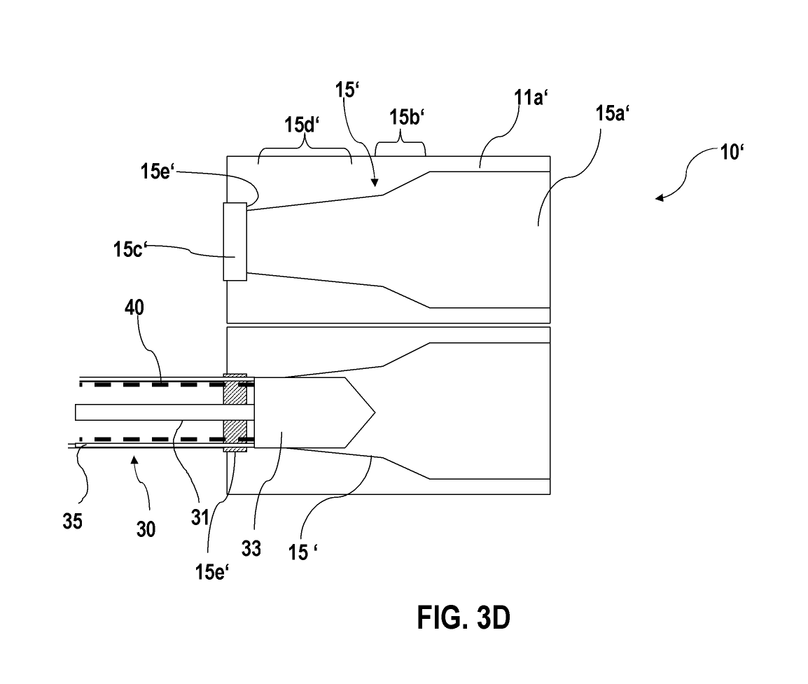

FIGS. 3A to 3D show schematic illustrations of a crimping device, which is modified as compared to the design according to FIGS. 1 and 2, during use, more specifically in FIG. 3A in the open state with the introduced insertion catheter, in FIG. 3B in the closed state with the pushing tool placed against the forming tool, in FIG. 3C after completion of the mounting process, and in FIG. 3D in the folded-open state again for removal of the finished product. It is expressly pointed out that not all the figures show all parts of the insertion catheter and the illustrations of the pushing tool and stents (specifically as far as the lengths thereof are concerned) in FIGS. 3B and 3C are not entirely consistent with each other. FIGS. 3B and 3C, although they show the forming tool in the closed state, show the forming cavity similarly as with the tool being open.

The modified forming tool 10′ is composed of two hingedly attached half shells 11a′, 11b′ so that it can be folded open and defines a forming cavity 15′ comprising a cylindrical shaped stent receiving section 15a′ and an annular rim shaped outer tube receiving section 15c′. Hinges 13 for hingedly attaching the half shells 11a′ and 11b′ may be provided in a variety of different configurations as are general known. The tube receiving section 15c′ has a larger diameter than the adjacent diameter of the tapering section to define an annular shoulder that separates the receiving section 15c′ from the tapering section. The tapering section thereof is composed of two truncated cone sections 15b′ and 15d′ arranged sequentially to define a steadily narrowing diameter moving in an axial direction toward the outer tube receiving section 15c′. As indicated, the rate of change of the diameter varies between sections 15b′ and 15d′. In addition to the annular retaining and handling section 21′, the modified pushing tool 20′ comprises a stent forming section 23′, which comprises a larger number of metal rods 25 than the first embodiment.

An insertion catheter 30 is introduced in the forming tool 10′, the catheter comprising an inner tube 31 having a tip 33 and an outer tube 35 that can be axially displaced with respect to the same. The insertion catheter 30 is placed in the forming tool such that the distal end of the outer tube 35 ends up in the outer tube receiving section 15c′. Because the diameter of this section is greater than the diameter of the tapering section 15b′, 15d′ at the end, an annular shoulder or stop 15e′ is formed there, against which the distal end of the outer tube 35 strikes and is thereby received. The inner tube 31 with the tip 33 is pulled distally out of the outer tube that is fixed in this way, so that the annular space between the inner and outer tubes 31, 35 becomes accessible.

FIG. 3B shows how a vascular stent 40 is introduced in the stent receiving section 15a′ in the expanded state. The pushing tool 20′ is placed on from the open end of the stent receiving section 15a′ and pushed toward the opposing end of the forming tool 10′, which is to say in the direction of the distal end of the outer tube 35 of the insertion catheter 30. The ends of the rods 25 of the pushing tool 20′ engage in the struts of the vascular stent 40 that are located the furthest proximally (not shown) and carry these along (and thus the entire stent, deforming it) in the direction of the pushing movement. The stent is thus pushed through the two-piece tapering section 15b′, 15d′, wherein the diameter of the stent is reduced to the final dimension intended for the implantation process. At the same time, the stent is pushed into the annular space between the inner and outer tubes of the insertion catheter 30. The stent 40 ultimately assumes the position shown in FIG. 3C in the insertion catheter. This is the state ready for use. FIG. 3D shows the forming tool 10′ in the re-opened state for removal of the insertion catheter with the introduced stent.

The implementation of the invention is not limited to the examples described above and designs emphasized, but is likewise possible in a plurality of modifications, which are within the scope of standard practice in the art.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

What is claimed is:

1. A crimping device for mounting a vascular stent, or a heart valve prosthesis introduced in the vascular stent, on an insertion catheter, comprising:
a forming tool having a rotationally symmetrical forming cavity, which at one end comprises a stent receiving section, at the opposing end a stent outfeed section and in between the stent receiving section and the stent outfeed section, a tapering section, and
a pushing tool, which comprises a retaining and handling section and a stent forming section, wherein the stent forming section is designed to penetrate the forming cavity of the forming tool from an open end of the stent receiving section and to seize and push back the vascular stent introduced in the forming tool through the tapering section.

2. The crimping device according to claim 1, wherein the stent forming section of the pushing tool comprises a plurality of flexible rods, or ribs that are tapered toward free ends of the plurality of flexible rods or ribs, and the retaining and handling section comprises a circular ring-shaped or disk-shaped holder, on a circumference of which the rods or ribs are held on one side, in particular at identical angular distances.

3. The crimping device according to claim 1, wherein the forming tool has a two-piece design that can be folded open.

4. The crimping device according to claim 1, wherein the stent outfeed section is also designed as an outer tube receiving section that receives and fixes an outer tube of the insertion catheter.

5. The crimping device according to claim 4, wherein the outer tube receiving section of the forming tool has a larger diameter than an adjoining end of the tapering section such that an end of the outer tube receiving section forms an annular stop for a distal end of the outer tube of the insertion catheter.

6. The crimping device according to claim 1, wherein the tapering section of the forming tool has a truncated cone shape.

7. The crimping tool according to claim 1, wherein the tapering section of the forming tool is composed of at least two truncated cone-shaped sections having differing cone angles.

8. The crimping device according to claim 1, wherein the wall of the tapering section of the forming tool is curved.

9. The crimping device according to claim 1, wherein the forming tool has at least one section made of transparent material.

10. The crimping device according to claim 1, wherein the forming tool further comprises contact surfaces with a hydrophobic or hydrophilic coating.

11. A method for mounting a vascular stent, or a heart valve prosthesis introduced in the vascular stent, on an insertion catheter, comprising:
providing an expanded vascular stent or a heart valve prosthesis comprising an expanded vascular stent;
providing an insertion catheter comprising an inner tube having a catheter tip and an outer tube that is displaced with respect to the inner tube;
providing a forming tool having a rotationally symmetrical forming cavity, which at one end comprises a stent receiving section, at an opposing end comprises an outer tube receiving section and in between the stent receiving section and the outer tube receiving section, a tapering section;
providing a pushing tool, which comprises a retaining and handling section and a stent forming section, wherein the stent forming section is designed to penetrate the forming cavity of the forming tool from an open end of the stent receiving section and to seize and push back the vascular stent introduced in the forming tool through the tapering section;
introducing the vascular stent or the heart valve prosthesis in the stent receiving section of the forming tool;
introducing a distal end of the insertion catheter in the forming tool, wherein a distal end of the outer tube, in a retracted state, is fixed in the outer tube receiving section of the forming tool; and
inserting the stent forming section of the pushing tool into an open end of the stent receiving section of the forming tool and pushing back the vascular stent or the heart valve prosthesis through the forming cavity while reducing a diameter of the vascular stent or the heart valve prosthesis, and while simultaneously pushing the vascular stent or the heart valve prosthesis between the outer tube and inner tube of the insertion catheter.

12. A crimping device for mounting an implant on an insertion catheter, the device comprising:
a first half shell with a first side;
a second half shell that cooperates with the first half shell, the second half shell having a first side;
at least one hinge arranged about the first half shell first side and the second half shell first side to thereby pivotably connect the first and second half shells wherein they pivotably move about the at least one hinge and have a closed position wherein they abut one another; and,
a forming cavity defined between the first half shell and the second half shell when they are in the closed position, the forming cavity defining an outer tube receiving section at an open first end, a funnel shaped tapered section adjacent to the tube receiving section, an annular shoulder separating the outer tube receiving section from the adjacent funnel shaped tapered section, and a stent receiving section adjacent to the at least one tapered section and open at a second cavity end.

13. The crimping device according to claim 12 and further comprising:
a pushing tool having a retaining and handling section and a stent forming section, wherein the retaining and handling section is formed as a circular ring or disk, and the stent forming section is formed by a plurality of flexible rods that are attached on one side to the retaining and handling section at identical angular distances, the plurality of flexible rods configured for insertion through the stent receiving section; and,
wherein the forming cavity is configured to receive the pushing tool inserted into the stent receiving section and pushed toward the tube receiving section.

14. The device according to claim 13, wherein the plurality of flexible rods are tapered toward a free end of the rods, and wherein the forming tool permits visual control of a mounting or crimping process without interim opening of the forming tool.

15. A crimping device according to claim 12 wherein:
the funnel shaped tapered section comprises first and second truncated cone sections, the first truncated cone section having a first diameter that is smaller than the diameter of the tube receiving section to thereby define the annular shoulder between the tube receiving section and the tapered section, the first truncated cone section having an increasing diameter moving along its axial direction away from the tube receiving section;
the second truncated cone section adjacent to the first truncated cone section and having a diameter that is larger than the largest diameter of the first truncated cone section, the second truncated cone section having a gradually increasing diameter moving along its axis in a direction away from the first truncated cone section; and,
wherein the stent receiving section has a cylindrical shape with a constant diameter that is equal to the largest diameter of the second truncated cone section.

16. A crimping device according to claim 15 wherein the second truncated cone section has a different rate of varying diameter along its axis as compared to the first truncated cone section.

17. The device according to claim 12, wherein the device is configured to receive an insertion catheter having an inner tube, a tip, and an outer tube that are axially displaced with respect to each other, wherein the insertion catheter is placed in the forming tool such that one end of the outer tube is received in the outer tube receiving section.

18. The device according to claim 12, wherein the forming cavity is configured to reduce a diameter of the stent as it is pushed through the forming cavity to a final dimension intended for an implantation process.

19. The device according to claim 12, wherein:
the tapering section comprises a first truncated cone section and a second truncated cone section, the first truncated cone section having a first diameter that is greater than a diameter of the second truncated cone section, the first and second truncated cone sections arranged in sequence to define an increasing diameter moving in an axial direction from the tube receiving section towards the stent receiving section, and, the outer tube receiving section having a diameter that is greater than a diameter of the adjacent second truncated cone section to thereby define the annular shoulder that is configured to receive an outer tube of an insertion catheter.
